(19) United States
(12) Patent Application Publication
(10) Pub. No.: US 2025/0339639 A1
(43) Pub. Date: Nov. 6, 2025

(54) BREATHING SYSTEM FOR SUBSTANCE INHALATION

(71) Applicant: Inspired Ventilation Ltd, London (GB)

(72) Inventor: Alastair Rupert Joseph DARWOOD, London (GB)

(21) Appl. No.: 18/866,460

(22) PCT Filed: May 19, 2023
(86) PCT No.: PCT/EP2023/063537

§ 371 Date: Nov. 15, 2024

(30) Foreign Application Priority Data
May 18, 2022 (GB) 2207302.7

(51) Int. Cl. A61M 16/18; A61M 11/04; A61M 16/06; A61M 16/20

(52) U.S. Cl. CPC A61M 16/18; A61M 11/04; A61M 16/06; A61M 16/209; A61M 2205/3334

(57) ABSTRACT

A breathing system arranged for a patient to breath through continuously and to inhale a substance from during breathing, comprising: adding means for adding the substance to gas to be inhaled, and control means for pressurising contained gas. The adding means comprises: a reservoir for containing the substance in a liquid form; and vaporisation means for vaporising the substance. The contained gas is linked to the substance in the reservoir to drive the substance to the vaporisation means. The control means comprises a pressure reduction means for enabling loss of the pressurised gas at a rate that is less than a rate at which the control means can pressurise the gas.

### FIELD OF THE INVENTION

The invention relates to a breathing system for a patient to inhale a substance from, in particular an analgesic, anaesthetic and/or sedative substance. The invention also relates to a method of use of such apparatus.

### BACKGROUND

The inventor noted lack of a breathing system enabling inhalation of anaesthetic, analgesic and/or sedative substance by a patient in which the patient could influence or control the amount of anaesthetic, analgesic and/or sedative substance administered, so as to remain comfortable without intervention of a medical professional. Safety is a significant problem in such a system, to avoid risk of overdose. Also, conventional pumps for reliably adding small quantities of between 10 and 150 microlitres of substance to gas to be inhaled may be expensive. It is an object of the present invention to address these problems, wholly or in part.

### SUMMARY OF THE INVENTION

According to a first aspect of the present invention, there is provided a breathing system for a patient to breath through continuously and to inhale a substance from during breathing, comprising: adding means for adding the substance to gas to be inhaled, comprising a reservoir for containing the substance in a liquid form, and vaporisation means for vaporising of the substance; and control means for pressurising contained gas, wherein the contained gas is linked to the substance in the reservoir to drive the substance to the vaporisation means, the control means comprising a pressure reduction means for enabling loss of the pressurised gas at a rate that is less than a rate at which the control means can pressurise the gas. Thus, the pressure reduction means causes the amount of substance being added to gas to be inhaled to diminish as a function of time until no more of the substance is added.

The control means may further comprise a flow control means arranged for the substance to flow through between the reservoir and the vaporisation means and to restrict flow to the vaporisation means, wherein the contained gas is linked to the substance in the reservoir to drive the substance to the vaporisation means through the flow control means.

According to a second aspect of the present invention, there is provided a breathing system for a patient to breath through continuously and to inhale a substance from during breathing, comprising: adding means for adding the substance to gas to be inhaled, comprising a reservoir for containing the substance in liquid or gaseous form, and vaporisation means for vaporising the substance; and control means for pressurising contained gas, wherein the contained gas is linked to the substance in the reservoir to drive the substance to the vaporisation means, the control means comprising a flow control means arranged for the substance to flow through between the reservoir and the vaporisation means and to restrict the flow.

The flow control means may be located in a tube between the reservoir and the vaporisation means through which the substance flows or at an end of said tube.

The control means may further comprise a pressure reduction means for enabling loss of the pressured gas at a rate that is less than a rate at which the pressurising means can pressurise the gas.

The following features are optional and/or preferred features of the first and/or second aspects.

The pressure reduction means may include a rate control for adjusting the rate. For example, the pressure reduction means may be a needle valve enabling such control.

The control means may include a user control operable by a patient to provide inputs to the control means further to operations by the patient. The user control may be operable by the patient by a consciously performed action, for example a finger or hand, blink or bite action.

The pressure reduction means may be coupled to the control means and the control means may be configured to adjust the rate of loss of pressurised gas dependent at least on the inputs.

The control means may be configured to control the pressurising of the contained gas dependent at least on the inputs.

The inputs may be pneumatic, mechanical or electrical.

The user control may be located, in use of the breathing system, spaced from the patient airway interface, for example for holding or wearing by a hand of the patient. One or more further parts or a whole of the control means may be located, in use of the breathing system, spaced from the patient airway interface. In addition, the reservoir may be located spaced from the vaporising means and connected thereto with the conduit for provision of the substance to the vaporising means. The reservoir may be located with the user control as part of a hand held device.

The user control may be operable by the patient when in a first state (for example an expanded state where the inputs are pneumatic) and operation thereof moves the user control to a second state (for example a collapsed state where the inputs are pneumatic). The control means may in this case include a restitution limiter configured to set a time period at which the user control returns to the first state from the second state.

The user control may be biased to the first state, and may comprise means for biasing to the first state.

Operation of the user control to move the user control from the first state to the second state may remove air from a chamber within the user control. In this case, the restitution limiter permits air intake into the chamber at a rate such that the user control returns to the first state at the end of the time period.

The control means may include an expansile member into which gas is located by operation of the user control, thereby to expand the expansile member. The expansile member is biased to a collapsed state. In this case the expansile member may act on the substance to drive the substance to the vaporisation means.

Alternatively, the control means may further comprise pressurising means, for example an air compressor, for pressurising the gas, and a controller coupled to the pressurising means for controlling the pressurising means to control the pressurising of the gas. For example, the controller may provide control instructions to the pressurising means. The user control and the controller may be electrically connected and the inputs are received by the controller are electrical.

The contained gas may be linked to the substance in the reservoir via an intermediary member that the contained gas causes to push against the substance, thereby to drive the substance to the vaporisation means. The intermediary member may comprise a resiliently deformable membrane separating the gas and the substance. The membrane may be an expansile membrane sealingly attached to the reservoir around an opening of the reservoir connecting the reservoir and a gas supply, arranged so that, when gas pressure inside the expansile membrane increases, the expansile member expands into the substance.

The breathing system may further comprise: a patient airway interface mountable on the patient so that inhaling and exhaling occur through the patient airway interface repetitively and consecutively.

The patient airway interface may be mountable on the patient to be retained in place indefinitely without use of hands of the patient. For example, the patient airway interface may be mounted on the patient by a strap around the patient's head.

In use of the breathing system, the vaporising means or the adding unit may be fixedly mounted on the patient airway interface. The adding means may not be carried other than by the patient airway interface.

The control means, other than the user control, may be fixedly mounted on the patient airway interface.

The substance may be an analgesic, anaesthetic and/or sedative substance.

The components required to make the breathing system may be obtained and assembled at reasonable cost. Although not essential, the breathing system may be single-use.

### BRIEF DESCRIPTION OF DRAWINGS

Embodiments of the invention are described in the following, by way of example only, with reference to the accompanying Figures in which:

FIG. 1A shows illustratively components in accordance with embodiments of the invention;

FIG. 1B is a block diagram of components of an adding unit and control apparatus in accordance with an embodiment;

FIG. 2A is an illustration of components of an adding unit in accordance with another embodiment;

FIG. 2B is an illustration of the components of FIG. 2A, and additionally a housing;

FIG. 3 is an illustration of the components of FIG. 2A, and additionally a coupled expansile member in accordance with a modified embodiment;

FIG. 4 shows illustratively components of a breathing system in accordance with another embodiment;

FIG. 5 shows illustratively components of a breathing system in accordance with a yet further embodiment;

FIG. 6 shows illustratively components of a breathing system in accordance with another embodiment similar to that of the embodiment of FIGS. 2A and 2B, but with a single control for operation of two components;

FIG. 7 is an illustrative cross-sectional view of a patient face mask for use in embodiments;

FIG. 8A is an illustration of an embodiment including an intermediary member between contained gas and a substance;

FIG. 8B is an illustration of an embodiment including a variant intermediary member;

FIG. 8C is an illustration of an embodiment including a further variant intermediary member in the form of a piston.

### DETAILED DESCRIPTION OF EMBODIMENTS

Embodiments of the invention relate to a breathing system for controllably adding a substance to a gas for inhalation. Herein references to an “operator” are to be understood as being to the patient or to another person, such as a medical professional.

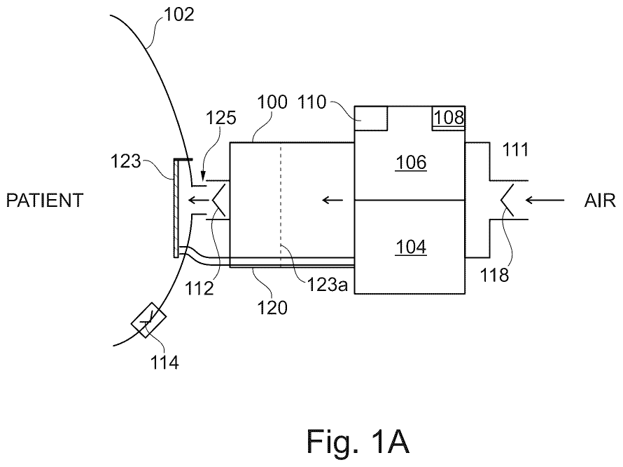

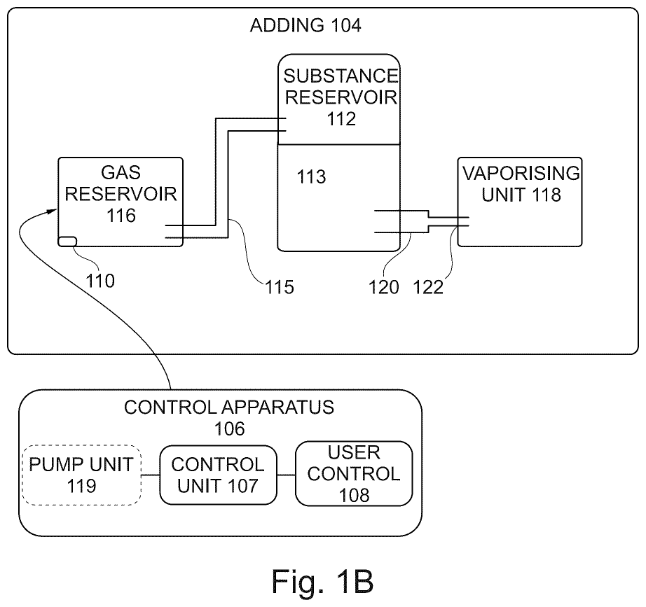

Referring to FIG. 1A, a breathing system in accordance with such embodiments includes a housing 100 with flow control valves, a patient airway interface (PAI) 102, an adding unit 104 and control apparatus 106. The control apparatus 106 includes a user control 108 and a pressure reduction component 110. In some embodiments, the control apparatus 106 includes one or more sensors. The control apparatus includes a pressure generation function to provide pressure to drive the adding of the substance. Referring also to FIG. 1B, the adding unit 104 comprises a substance reservoir 112, a first conduit 115, a gas reservoir 116, a vaporising unit 118, a second conduit 120 and a flow control means in the form of a flow restrictor 122. The control apparatus includes a pump unit 119 shown in broken lines to indicate that in some embodiments the pump unit 119 may be absent with the pressure generation functionality included in a control unit 107 and, in some of those embodiments, also the user control 108.

The housing 100 provides a structure for mounting of the PAI 102, the adding unit 104 and the control unit 107. The PAI 102 is initially disconnected from the housing 100 and is connectable to the housing 100 for use. A clipping system on the PAI 102 and the housing 100 not shown in the Figures but whose location is indicated at 125 is provided. Alternatively the PAI 102 and the housing 100 may be connected at the same location using corresponding screw threads. Embodiments are not limited to any particular way in which the PAI 102 and the housing 100 are connected. Initial disconnection enables different shaped/sized PAIs to be selected for different patients, as well as disposal of the PAI 102 after use and re-use of the rest of the breathing system. Alternatively, the PAI 102 and the housing 100 may be provided as a single component, that is, be permanently connected.

Where the PAI 102 and housing 10 are connected, they are fixedly disposed relative to one another. The adding unit 104 and the control apparatus 106 are mounted on the housing 100 and are fixed relative thereto. In variant embodiments, such parts may be flexible attached. In some variant embodiments, one or more parts of the adding unit 104 (e.g. the substance reservoir and/or the gas reservoir 116) and/or one or more parts (e.g. the user control 108) or all of the control apparatus 106 may not be mounted on the housing 100 and may instead be connected to other of the parts mounted on the housing 100, for example via a flexible cable. In particular, as described below the user control 108 can be physically spaced from the rest of the control apparatus 107 and may, for example, be hand-held and connected to another part of the control apparatus with such a cable. In some embodiments, the control apparatus may be provided as a hand-held device operable by the patient. Such a device may also include the substance reservoir 112 and/or the gas reservoir 116, in which case the second conduit extends between the hand-held device and the vaporising unit 118.

The flow control valves include first and second one-way inspiratory valves 118, 112 and a one-way expiratory valve 114. The first inspiratory valve 118 is located at an intake 111 to the breathing system and prevents escape of gas with the substance in through the intake 111 to the environment. The second inspiratory valve 112 is located to prevent exhaled gas from entering the vaporisation unit 118. In some embodiments, the second inspiratory valve 112 is optional and may be omitted. The expiratory valve 114 is located to enable exhaled gas to exit the breathing system and to prevent air from entering from the environment. Arrows are present in FIG. 1 indicating gas flow through the breathing system. A filter (not shown), such as a charcoal filter, may be located with the expiratory valve 114 to remove any residual substance in gas flowing out of the PAI 102.

Referring also to FIG. 1B, the substance reservoir 112 contains the substance 113 which is in a liquid form, and also gas above a surface of the substance. The gas reservoir 116 also contains the gas. The gas is air, although in variant embodiments one or more other gases may additionally or alternatively be used. The substance reservoir 112 is operatively coupled to the gas reservoir 116 by the first conduit 115. The pressure of the substance in the substance reservoir 112 equalises with the pressure of the gas in the gas reservoir 116. When the breathing system is in use, the substance reservoir 112 is orientated such that an outlet of the conduit 115 into the substance reservoir 112 is located above the surface of the substance 113. The control apparatus is operatively coupled to the gas reservoir 116 so as to increase pressure of the gas contained in the gas reservoir 116.

The substance reservoir 112 is coupled to the vaporisation unit 118 by the second conduit 120 and the flow control 122. Substance flowing from the substance reservoir 112 to the vaporisation unit 118 flows through the second conduit 120 and the flow control 122. The pressurised gas acts on the substance to drive the substance through the second conduit 120 and the flow control 122. The flow control 122 is arranged to restrict the flow rate of the substance out of the reservoir 112. For example, the flow rate may in use be restricted by the flow control 122 to less than 10 microlitres per second, or less than 5 microlitres per second, or less than 2 microlitres per second. The flow rate may be dependent on a pressure difference across the flow control, but the apparatus may be configured so that the flow rate does not exceed these limits.

The flow control 122 may be a hollow tube for example in the form of a needle. Alternatively, the flow control may provide a labyrinthine flow passage. Alternatively, the flow control may comprise a region of compacted or woven material such as a wick or compact fabric. In variant embodiments, the flow control 122 is not limited to these and alternatives may be provided that function to hinder free flow of the substance 113 to the vaporisation unit 118, such that a driving pressure is required to generate clinically significant substance flow.

In variant embodiments where the gas reservoir 116 and the substance reservoir 116 are adjacent, the first conduit 115 may be in the form of an opening between the gas reservoir 116 and the substance reservoir 112. Also, in variant embodiments where the substance reservoir 116 and the vaporising unit 118 are adjacent, the second conduit 120 may simply consist of the flow restrictor 122 with an opening to the substance reservoir 112 and the vaporising unit 118.

The control apparatus comprises a control unit 107 as well as the user control 108. The user control 108 is operable to provide inputs to the control unit 107 in response to operations of the user control 108 by the operator. The user control 108 is operable in a manual action for example. While the user control 108 may be mounted on the housing 100, in preferred embodiments, the user control 108 is coupled to other control components of the control unit 107 by a flexible cable, so that the user control 108 can rest or be held in a patient's hand for easy manual operation, that is, be hand-held and/or wearable. In variant embodiments the user control 108 is operable in any physical action that can be performed by the patient with conscious control, for example a bite action or a blinking action. Alternatively, the user control 108 may be disposed for operation by an operator other than the patient.

In some embodiments the control unit 107 includes a microcontroller, and the pressure generation function includes a pump unit 119. Where this is the case, the microcontroller is coupled to the user control 108 and the user control 108 is configured to provide electric signals to the microcontroller in response to operation by the patient. The microcontroller may also be coupled to the one or more sensors to receive data from the one or more sensors. The microcontroller is also coupled to the pump unit 119 to control pressurising of gas in the gas reservoir 116 by the adding unit.

The microcontroller includes a processor and a memory. The memory has stored on it computer program code executable by the processor for determining control instructions for the pump unit 119 dependent on the received inputs and, where the one or more sensors are present, real-time data from the one or more sensors. In variant embodiments, to perform the determining, the control unit 107 may be provided with dedicated hardware or a mixture of dedicated hardware and software. The control apparatus may in other embodiments not include a microcontroller and may be exclusively mechanical and/or pneumatic in nature, that is, have exclusively mechanical and/or pneumatic control components.

The pressure reduction component 110 permits loss of gas from the gas reservoir 116 to the environment at a rate that is less than the rate at which the pump unit 119 can add gas. For example, the pump unit 119 may pressurise gas in the gas reservoir in less than one second, while the pressure reduction component 110 may result in loss of sufficient pressure in the gas reservoir 116 to result in adding of the substance to gas to be inhaled in greater than 2 s but less than 60 s or less than 30 s or less than 15 s or less than 10 s. The pressure reduction component 110 is thus configured to cause gradual decrease in the pressure of the gas when the pump unit 119 is not operated to increase the gas pressure. The pressure reduction component 110 may be configured to allow escape of contained gas to the environment until remaining contained gas is at environmental pressure. Alternatively, the pressure reduction component 110 is configured to permit decrease in pressure in the reservoir 112 until the pressure has decreased to a predetermined pressure lower than the pressure needed for the substance to flow through the flow control 122 but greater than environmental pressure. The pressure reduction component 110 may be in the form of a valve in a wall of the gas reservoir 116 connecting an interior of the gas reservoir 116 with the exterior, and may be referred to n as a “leak valve”.

The pressure reduction component 110 may be configured to permit escape of gas from within the gas reservoir 116 at a fixed rate. In a variant embodiment, the rate may instead be dependent on the pressure within the gas reservoir 116. In another variant embodiment, the rate may be settable by the operator; for example the control apparatus 106 may include a rate control enabling this. In some embodiments, the rate may be dependent on inputs caused by operations of the user control 108 by the patient. In such embodiment, where the control unit 106 includes a microcontroller, the pressure reduction component 110 may be operatively connected to the microcontroller and the microcontroller configured to control the rate dynamically, using predetermined algorithms of the code, which cause change of the rate dependent on the inputs from operation of the user control 108. In other variant embodiments also where the control apparatus includes such a microcontroller, such predetermined algorithms may cause change of the rate dependent on real-time information received from the one or more sensors, additionally or alternatively to such inputs. The higher the rate, the sooner the adding of substance to inhaled gas ends in absence of further pressurisation by the pump unit 119.

In a variant embodiment, the pressure reduction component 110 is located in the first conduit 115 rather than in the gas reservoir 116. In another variant embodiment, the pressure reduction component 110 is instead located in the substance reservoir 112 above the surface of the substance. In this case, the pressure reduction component 110 is located such that, in use of the breathing system, the pressure reduction component 110 is disposed to have gas pressing against it rather than the substance, that is, be above the surface of the substance 113.

The PAI 102 is illustrated in the form of a face mask. In variant embodiments, the mask 10 may be replaced with another type of PAI. The PAI may be in the form of a nasal clip, nasal mask or an invasive device such as a supraglottic airway, for example. Such a nasal mask may, for example, be of particular application in the field of dentistry. Breathing through the mouth may be avoided by the patient or prevented by means known in the field. Embodiments of the invention are not limited to any particular form of PAI 102 that acts as a conduit for all inhaled and exhaled respiratory gasses and that requires the patient to repetitively and consecutively inhale and exhale through the breathing system. For example, in many applications the PAI may be mounted on the patient such that inhalation and exhalation occur exclusively through the breathing system for at least 10 minutes. For many applications, the PAI may be mounted in such a way for at least 30 minutes.

The vaporising unit 118 comprises a material 123 onto which the substance is dispensed after exit from the flow control 122. The vaporising unit may comprise exclusively of the material 123. The material 123 is attached across an interior of the PAI 102, being attached at edges to the PAI 102, so that entrained gas must pass through it. The second conduit 120 extends into the PAI 102 to dispense on the material 123. This may require insertion of the second conduit 120 through an aperture in the PAI 102 if the housing 100 and the PAI 102 are initially detached. In alternative embodiments the material may instead be attached across an interior of the housing 100, or includes a portion of the housing 100, the location of the material being indicated in this case by the broken lines 123a. In some embodiments, the vaporising unit 118 may include a grate mounted on the housing 100 against which the material 123 is located, or two grates between which the material 123 is sandwiched.

The material 123 is preferably configured to spread the substance, for example, in a wicking action, to increase the surface area of the substance and thereby facilitate evaporation into passing air. The material is preferably absorbent, although embodiments are not limited to such. The material 123 is arranged to span perpendicularly with respect to a flow path of entrained gas. Entrained gas is required to pass through the material 123, which presents low resistance to inhalation. In variant embodiments, the material 123 may span only in part across the housing rather than wholly, or extend lengthwise with respect to the direction of air flow, such that entrained air passes over it. Embodiments are not limited to any particular disposition of the material 123 whether located in the housing 100 or in the PAI 102. The material 123 may, for example, be in the form of a piece of gauze or fabric (e.g. cotton) pad. Embodiments are not limited to any particular form.

The vaporising unit 118 is configured to spread the substance so that it evaporates quickly in view of the rate at which the substance flows to the vaporising unit 118. This avoids retention of the substance on the material 123 for periods that are long in view of the breathing rate of the patient, e.g. greater than 10 s. Also, the vaporising unit 118 is configured to facilitate quick evaporation to avoid any pooling of the substance in housing 100.

In use, the breathing system enables the patient to breathe through it and also administers the substance, typically further to inputs from the operator. First, the PAI 201 is fitted to the patient such that the patient is compelled to breathe through the breathing system, with repetitive and consecutive inhalations and exhalations. An operator may be required to turn on the breathing system. The patient then operates (or attempts to operate in some embodiments below) the user control 108 when it is wanted for more substance to be administered. The control apparatus determines if gas in the gas reservoir 116 is to be pressurised, thus causing more substance to be added air to be inhaled, and if so the gas reservoir 116 is pressurised accordingly. Where the control apparatus includes the control unit 107 including the microcontroller, as well as the pump unit 119, this includes the microcontroller making the determination and providing control instructions to the pump unit 119 accordingly, the pump unit 119 acting to increase the pressure in the gas reservoir 116. Where the control apparatus is solely pneumatic and/or mechanical, the determination is dependent on the state of the control apparatus.

Embodiments described below can be considered to be possible implementations of the numerous generalised embodiments described above. Parts that are the same or have like functionality in different embodiments are denoted in the respective one or more Figures illustrating the respective embodiment by the same number incremented by 100 times the number of the corresponding Figure.

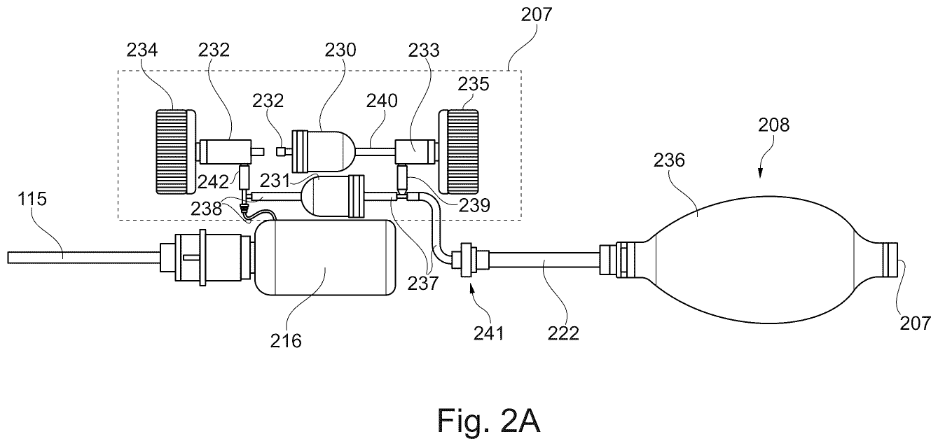

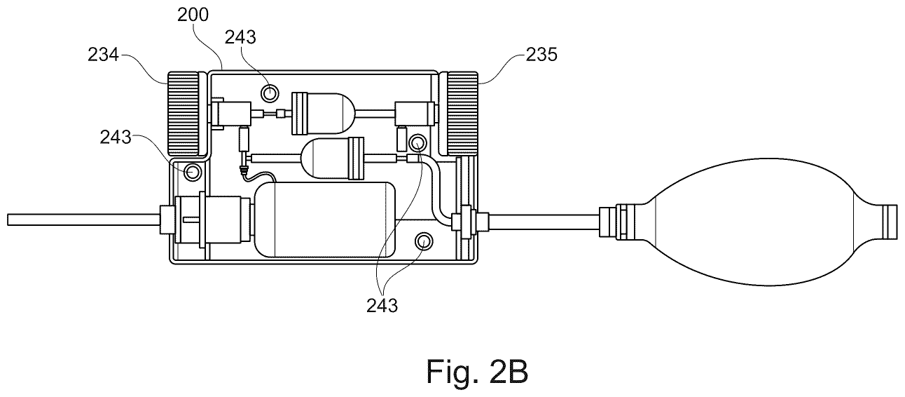

Referring to FIG. 2A, in accordance with an embodiment the control apparatus is mechanical and pneumatic. The control apparatus comprises a control unit 207 and a user control 208. The parts are operable to provide gas to the substance reservoir 112 described above, to drive substance to the vaporising unit 118.

The control unit 207 comprises first and second one way valves 230, 231, a pressure reduction component in the form of an adjustable first needle valve 232, a restitution time control in the form of an adjustable second needle valve 233, and tubes. The first needle valve 232 includes a first dial 234 for control of flow rate through the first needle valve 232. The second need valve 233 includes a second dial 235 for control of flow rate through the second need valve 233.

The first needle valve 232 is coupled to the gas reservoir 216 to permit gradual escape of contained gas. The first needle valve 232 is operable with the first dial 234 to adjust the rate of escape of contained gas, which also depends on pressure of the gas.

The user control 208 is in the form of a pump 236, which is preferably to be held in a hand—of the patient. A cable 222 is a tube sealingly connected to the pump 236. The pump 236 has an interior chamber and is squeezable in a manual squeeze action to provide an input in the form of pumped air to the control unit 207. The pump 236 is biased towards an expanded state; for example the pump 236 may be formed of suitable resilient rubber configured inherently to move to the expanded state. Alternatively, the pump 236 may be formed of a bellows or like device, biased to an expanded state by a resilient member such as a spring.

The cable 222 is connected to a first of the tubes 237 at a first connector 241 at one end thereof, which is in turn connected to the second one-way valve 231 at the other end thereof. A second of the tubes 238 connects the second one-way valve 231 to the gas reservoir 216. The second one-way valve 231 permits flow of air from the pump 236 to the gas reservoir 216, that is, from the first tube 237 to the second tube 238 and prevents flow in an opposite direction.

The second needle valve 233 is connected by a third of the tubes 239 to the first tube 237. The second needle valve 233 is also connected to the first one-way valve 230 by a fourth of the tubes 240. The first one-way valve 230 permits flow of environmental air into the second needle valve 233 and prevents flow of air in an opposite direction. The control unit 207 is thus arranged so that the pump 236 must take in air through the second needle valve 233 in order for the pump 236 to expand from a squeezed state to the expanded state. Thus, the restitution time control limits the rate at which the patient is able to cause the pump to supply to the control unit 207, thereby setting a maximum rate to the amount of substance that can be administered to the patient.

The first needle valve 232 is connected to the second tube 238 by a fifth tube 242. Air can flow from the gas reservoir 216 to the environment through the second needle valve 232.

Referring in addition to FIG. 2B, the parts are mounted on a housing 200. Bolts 243 keep sections of the housing 200 in place.

In use, the patient operates the user control 208, that is, squeezes the pump 236. This forces air through the cable 222, through the second one-way valve 231, thereby to pressurise the gas reservoir 216. The pressurised gas acts to drive the substance towards the flow control 122. Contemporaneously with the substance being so driven, air exits the control unit 207 through the first needle valve 232, thereby reducing the pressure in the gas reservoir 216. Also, since the pump 236 is biased to its expanded state, the pump 236 acts to cause intake of air through the second needle valve 233. The rate of return to the expanded state is dependent on the rate at which the second needle valve 233 permits air to flow through it.

In a variant embodiment, the gas reservoir 216 may be resiliently expandable. For example, the gas reservoir 216 may be formed of deformable, resilient material, such as rubber or latex. Alternatively, the gas reservoir 216 may be formed of expandable, flexible material and biased to an initial volume. Operation of the user control may cause expansion of the gas reservoir 216.

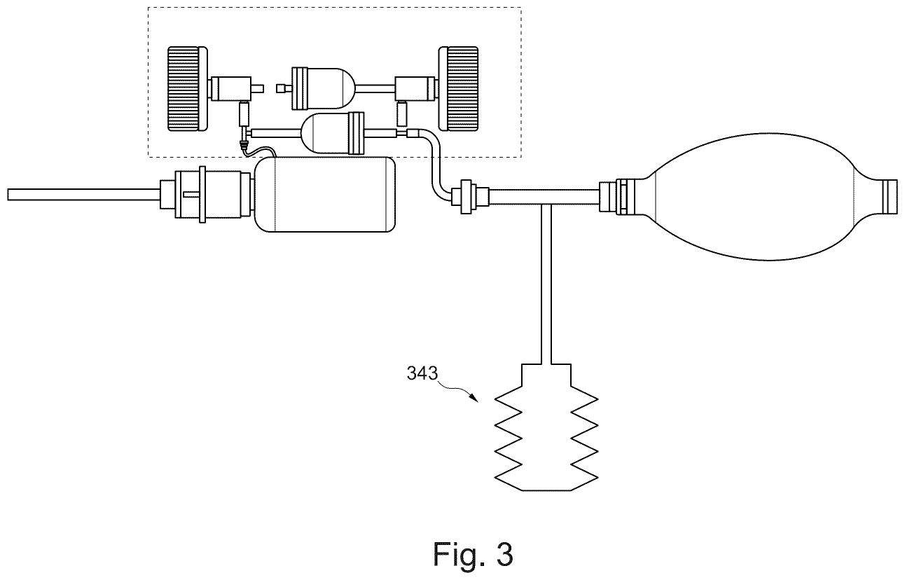

Referring to FIG. 3, the parts are the same as those in the embodiment shown in FIG. 2 or in the variants thereof, except, in addition, an expansile member 343 is connected to the cable 222. In variant embodiments, the expansile member 343 need not be connected to the cable 222 but may be connected anywhere with an interior of the expansile member 343 being in communication with an interior of the pump 236. For example, the expansile member 343 may be connected to the first or third tubes 237, 239, or directly to the pump 236, such as at a remote end thereof. The expansile member 343 can be inflated to an expanded state and is biased towards a collapsed state. Where the pump 236 is operated, air pressure is increased in the cable 222 and thus in the expansile member 343, thereby causing the expansile member 343 to expand. Inclusion of the expansile member 343 prevents a high peak pressure in the gas reservoir 216 following operation of the pump 236 and results in the pressure in the gas reservoir 216 being above the pressure required for flow of the substance to the vaporising unit 118 for longer, thus smoothing a profile of the pressure in the gas reservoir 216. The expansile member 343 is formed of rubber or latex, for example, configured inherently to move to a collapsed state. Alternatively, the expansile member 343 may be formed of a bellows or like device, biased to an collapsed state by a resilient member such as a spring.

In use, the parts function in the same way as those of FIG. 2, except that in addition air flows into the expansile member 343, causing expansion of the expansile member 343. The expansile member 343 acts to return to its contracted state.

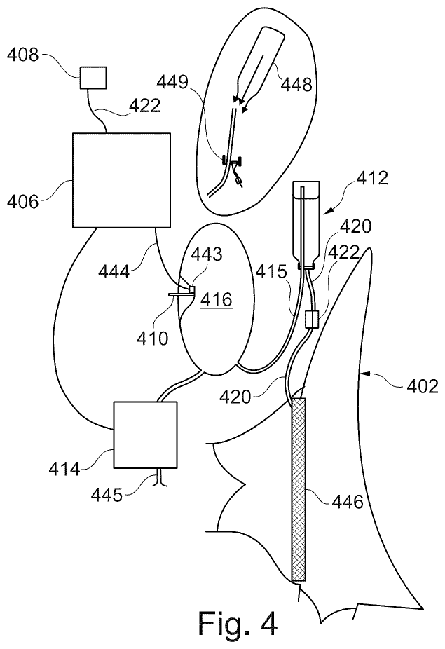

Referring to FIG. 4, in another embodiment, the pressure generation function in the form of pump unit 419 includes an air intake 445. The control unit 407 includes a microcontroller and the one or more sensors include a pressure sensor 443 electrically coupled to the microcontroller by cable 444. The user control 408, for example in the form of a button, is operable to provide inputs in the form of electrical signals to the microcontroller via the cable 422 in response to operations of the user control by the operator or patient.

The pressure sensor 443 provides data indicating real-time pressure in the gas reservoir 416 to the microcontroller. The microcontroller is configured to control the pump unit 414 dependent on the pressure in the gas reservoir 416, as well as dependent on inputs received from the user control 408 caused by the operator and on its stored algorithms. The pump unit 414 may be in the form of a compressor.

The substance reservoir 412 comprises a container 448 having a threaded opening at a base 449 thereof. The container 448 is for use in an orientation where the opening is at a bottom of the container. The reservoir 412 includes a closure member 449 threaded to thread onto the base 449, thereby to sealingly close the container 448. The closure member includes a first aperture therein through which the first conduit 415 sealingly extends. The closure member 449 also includes a second aperture to which an end of the second conduit 420 connects. An end of the first conduit 415 is located within the container 448 and terminates near a top of an interior of the container 448, so as to be above a surface of the substance. Substance is prevented from draining from the container 448 by the flow control 422 as well as by gas pressure in the gas reservoir 416. The flow control 422 permits the substance to flow from the container 448 when the pressure of the gas in the container 448 is greater than gas pressure at an end of the second conduit 420 at which substance is supplied to the vaporising unit, being the material 446 in the illustrated embodiment.

The control apparatus includes a leak valve 410 enabling gradual escape of gas from the gas reservoir 416. The leak valve 410 extends approximately from a mid-point of the gas reservoir 416 to reduce risk of the substance reaching the leak valve and potentially clogging it.

The container 448 may be a vial obtained from a supplier with the substance in and a non-shown seal.

In use, the control unit 407 receives inputs from the user control 408 further to operations of the user control by the patient. The control unit 407 determines whether to provide control instructions to the pump unit 414 to cause adding of the substance to gas to be inhaled. When the pump unit 414 is operated to increase the gas pressure, the substance flows to the vaporisation unit, through the second conduit 420. The leak valve 410 limits the time over which the gas reservoir 416 is sufficiently pressurised as to drive adding of the substance. The control unit 407 also receives real-time pressure data from the pressure sensor 416, such that the control unit 407 is provided with feedback. Thus a closed loop control may be achieved.

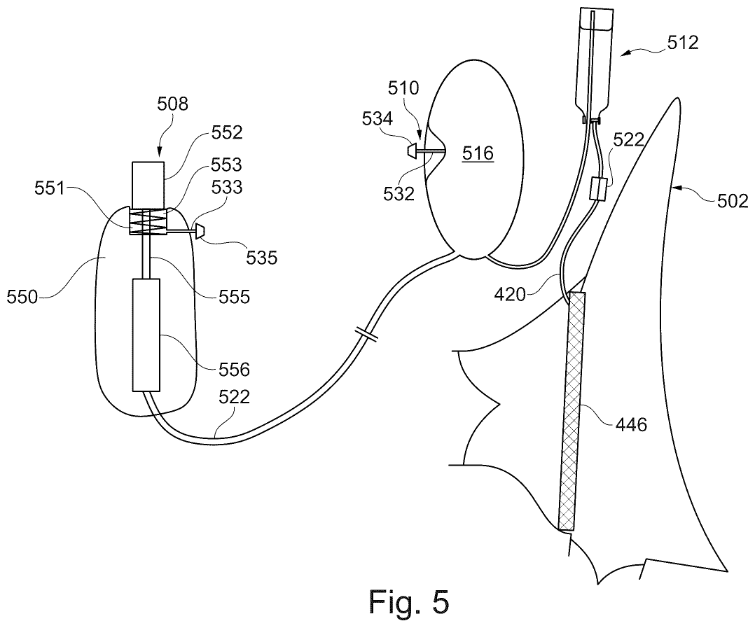

Referring to FIG. 5, in another embodiment the control apparatus includes in the user control 508 a body 550, the cable 522, a recess 551 in the body 550, a button 552, a spring 553, a restitution time control, a plunger 555 and a cylinder 556. The button 552 is moveable into the recess 551 (or chamber) into a depressed position (or state) by the patient against the spring 553, which biases the button 552 into an extended position. The plunger 555 is slidably located in the cylinder 556. The plunger 555 is attached to the button 552, such that depressing the button 552 into the recess 551 causes the plunger 555 to move into the cylinder 556. The cable 522 is sealingly connected to the cylinder 556 at one end thereof and at another end to the gas reservoir. Thus, depressing of the button causes air in the cylinder 556 to be pushed towards the gas reservoir, thereby to pressurise gas in the gas reservoir 516.

The restitution time control comprising a second needle valve 533 and second adjustment dial 535, the second needle valve 533 being between the recess 551 and the environment. These function as second needle valve 233 and adjustment dial 235. The second needle valve 233 limits the rate at which environmental air can enter the recess 551, thereby limiting the rate at which the button 552 can return to an extended position, thereby limiting the rate at which the patient can cause air to be provided to the gas reservoir 516.

The control apparatus includes a leak valve 510 comprising a first needle valve having a needle 532 and a control 534. The leak valve 510 extends approximately from a mid-point of the gas reservoir 516. Like the leak valve 410, the leak valve 510 limits the time over which the gas reservoir 416 is sufficiently pressurised as to drive adding of the substance.

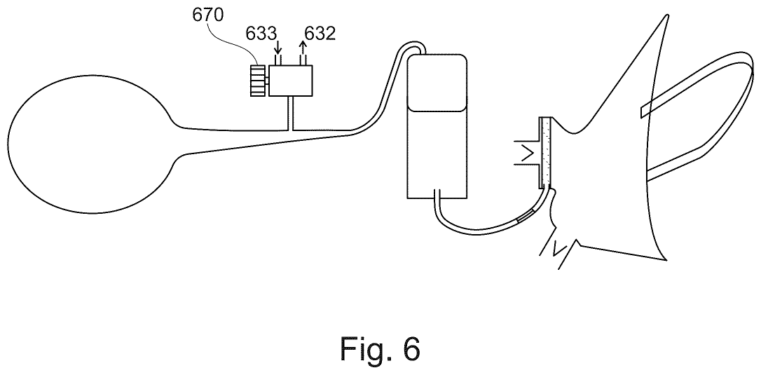

Referring to FIG. 6 which is simplified relative to FIG. 2A, in a variant on the embodiment described in relation to FIGS. 2A-2B, flow through the first and second needle valves 632, 633 may be controlled by a single control, such as a single dial 670. In this case, operation of the control causes air flow rate through the first and second needle valves 632, 633 to both reduce or both increase.

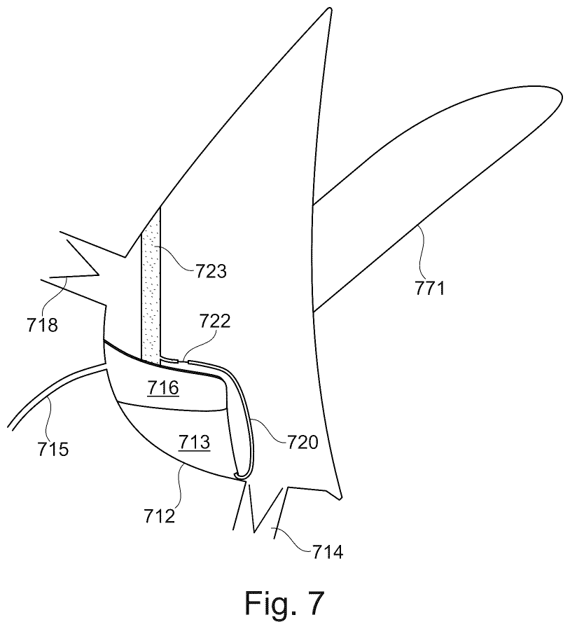

Referring to FIG. 7, a substance reservoir 712 is included with a face mask. A first conduit 715 extends from the face mask to couple to control apparatus for pressurising the gas. The face mask includes a strap 771 for mounting the mask to the patient's head. The material 723 is retained in mesh (not shown), the mesh being bonded at edges thereof to an interior of the face mask.

The first conduit 715 opens to the reservoir 712 in a vicinity of an uppermost part of the reservoir 712, the uppermost part being defined as such based on an orientation of the reservoir 712 when the face mask is mounted on a patient. The second conduit 720 opens to the reservoir 712 in a vicinity of a lowermost part thereof, the lowermost part being defined as such based on an orientation of the reservoir 712 when the face mask is mounted on a patient.

In preferred embodiments of the invention, the substance reservoir is oriented when the breathing system is in use such that contained liquid is at a base of the reservoir under the influence of gravity regardless of whether the patient is in an upright or supine position. In some embodiments, the gas reservoir may include a drainage valve. Thus, in the event that any substance accidentally entered the gas reservoir, it would exit via the drainage valve.

In the above embodiments, pressurised gas acts directly on the surface of the liquid substance to drive the substance through the flow restrictor. In variant embodiments, the gas may act on an intermediary member. Such an intermediary member also permits the substance to be in gaseous or liquid form.

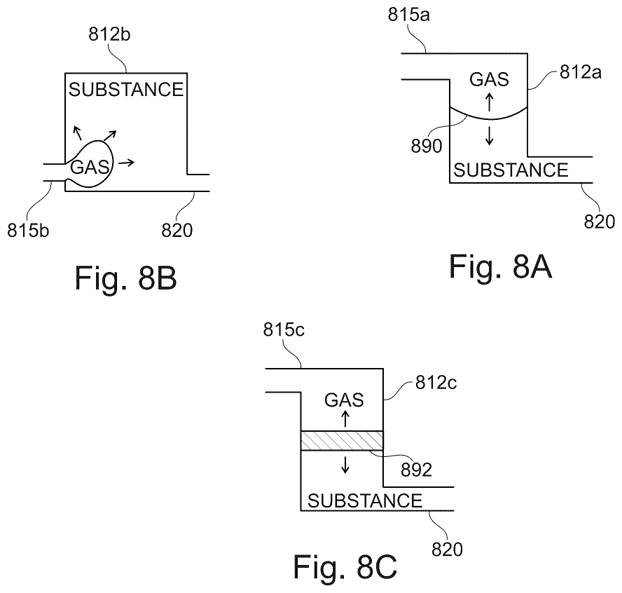

Referring to FIG. 8A, in one such embodiment, the intermediary member is in the form of a resilient flexible and/or deformable membrane 890 located between the gas and the substance. The membrane 890 is attached (by means of adhesive, for example) around an interior wall of the reservoir 812a to separate the gas and the substance. When pressure of the gas increases by entry of gas into the reservoir 812a through the first conduit 815a, it pushes the membrane 890 against the substance, driving the substance through the second conduit 820. Embodiments are not limited to any particular orientation of the membrane 890. The membrane 890 may be formed of rubber or latex, for example.

Referring to FIG. 8B, in another such embodiment, the intermediary member is in the form of an expansile bag 891 sealingly attached to an interior of the reservoir 812b around an opening of the first conduit 815b into the reservoir 812b. When the pressure of the gas increases in the bag, the expansile bag 891 expands against the substance. Embodiments are not limited to any particular disposition of the bag in the reservoir 812b, although the bag is preferably at least partially submerged in the substance. The bag 891 may be formed of rubber or latex, for example.

Referring to FIG. 8C, in another such embodiment, the intermediary member is in the form of a piston 892 and the reservoir 812c is, at least in part, a cylinder. The piston 892 is slidable in the reservoir. When the pressure of gas increases, the gas pushes the piston 892 against the substance, thereby to drive the substance towards the second conduit 820. The piston 892 may be formed of hard plastics material, for example. The plunger 555 described above is an example of a similar intermediary member in another embodiment.

The substance is a volatile liquid. The substance may be an analgesic, anaesthetic and/or sedative substance for having an analgesic, anaesthetic and/or sedative on a patient. For example, the substance may be methoxyflurane,, sevoflurane or ketamine. In variant embodiments the substance may be such as to have alternative or additional effects.

The substance reservoir of the embodiments may in all embodiments be in the form of a medical vial. In variant embodiments, alternative forms of reservoir may be provided that may be configured for topping up with the substance.

In alternative embodiments, the vaporising unit 118 is not limited to being the material. Alternative means for vaporising the substance may be provided. In some embodiments, particularly where the substance to be used is highly volatile, the vaporising unit 118 need not spread the substance to facilitate evaporation. Rather, the vaporising unit 118 may merely be a space into which the substance evaporates.

In alternative embodiments, the vaporising means may include a heater for heating the substance. The heater may be located at the outlet of the second conduit 120. In such cases, the substance need not be a volatile liquid and can be any substance that can be evaporated by heating at a temperature aligned with capability of the heater. Where the heater is included, the control means includes a microcontroller and the heater is coupled to the microcontroller, which controls operation of the heater by determining and providing control instructions to the heater. For example, the control instructions may include an “on” and an “off” instruction ”. The control instructions may also control degree of heating. The control means may include a temperature sensor for providing to the control means information indicative of temperature of the substance and/or ambient temperature, and the control means may determine control instructions as to degree of heating dependent on said information.

In alternative embodiments, the vaporising unit 118 may include a nozzle spray located at the outlet of the second conduit 120. In this case, the substance need not be a volatile liquid, but can be any liquid suitable for the nozzle spray to convert to droplets and that can be carried by gas within the breathing system and absorbed by a patient via inhalation.

Manufacturing processes for manufacture of the breathing systems in accordance with the above described embodiments will be apparent to persons skilled in the art. For example, the masks may be formed of a hard plastics material or silicone, and the airways of hard plastics material. Embodiments are not limited to use of specific materials.

Various modifications to the embodiments described above will be apparent to the skilled person. In some embodiments, the gas reservoir of the embodiments may be omitted. Instead, space above or otherwise adjacent the substance in the substance reservoir may act as a gas reservoir.

When used in this specification and claims, the terms “comprises” and “comprising” and variations thereof mean that the specified features, steps or integers are included. The terms are not to be interpreted to exclude the presence of other features, steps or components.

Unless otherwise stated, all individual features and/or steps of all embodiments described herein are disclosed in isolation and any combination of two or more such features is also disclosed, to the extent that such features or steps or combinations of features and/or steps are capable of being carried out based on the present specification as a whole in the light of the common general knowledge of a person skilled in the art, irrespective of whether such features or steps or combinations of features and/or steps solve any problems disclosed herein.

1. A breathing system arranged for a patient to breathe through continuously and to inhale a substance from during breathing, comprising:
a substance adder configured to add the substance to gas to be inhaled, comprising:
a reservoir for containing the substance in a liquid form;
a vaporiser configured to vaporise or facilitate vaporisation of the substance;
a control apparatus configured to pressurise contained gas, wherein the contained gas is linked to the substance in the reservoir to drive the substance to the vaporiser, the control apparatus comprising a pressure reduction valve for enabling loss of the pressurised gas at a rate that is less than a rate at which the control apparatus can pressurise the gas.

2. The breathing system of claim 1, wherein the control apparatus further comprises a flow restrictor arranged for the substance to flow through between the reservoir and the vaporiser and to restrict flow to the vaporiser.

3. A breathing system arranged for a patient to breathe through continuously and to inhale a substance from during breathing, comprising:
a substance adder configured to add for adding the substance to gas to be inhaled, comprising:
a reservoir for containing the substance in liquid or gaseous form;
a vaporiser for vaporising the substance;
a control apparatus configured to pressurise contained gas, wherein the contained gas is linked to the substance in the reservoir to push the substance from the reservoir to the vaporiser, the control apparatus comprising a flow restrictor arranged for the substance to flow through between the reservoir and the vaporiser and to restrict the flow.

4. The breathing system of claim 3, wherein the flow restrictor is located in a tube between the reservoir and the vaporise through which the substance flows or at an end of said tube.

5. The breathing system of claim 3, wherein the control apparatus further comprises a pressure reduction valve for enabling loss of the pressurised gas at a rate that is less than a rate at which the control apparatus can pressurise the gas.

6. The breathing system of claim 1, wherein the pressure reduction valve includes a rate control for adjusting the rate.

7. The breathing system of claim 1, wherein the control apparatus includes a user control operable by a patient to provide inputs to the control apparatus further to operations by the patient, wherein the inputs are pneumatic, mechanical or electrical.

8. The breathing system of claim 7, wherein the pressure reduction valve includes a rate control for adjusting the rate, wherein the pressure reduction valve is coupled to the control apparatus, and wherein the control apparatus is configured to adjust the rate of loss of pressurised gas dependent at least on the inputs from the user control.

9. The breathing system of claim 7, wherein the control apparatus is configured to control the pressurising of the contained gas dependent at least on the inputs; and/or
wherein the inputs are pneumatic, mechanical or electrical; and/or
wherein the user control is for manual operation located spaced from the patient airway interface.

10-11. (canceled)

12. The breathing system of claim 9, wherein the control apparatus is located, in use of the breathing system, spaced from the patient airway interface and/or wherein the reservoir is located spaced from the vaporiser and connected thereto with a conduit for provision of the substance to the vaporiser.

13. (canceled)

14. The breathing system of claim 7, wherein the user control is operable by the patient when in a first state and operation thereof moves the user control to a second state, wherein the control apparatus includes a restitution limiter configured to set a time period at which the user control returns to the first state from the second state.

15. The breathing system of claim 14, wherein operation of the user control to move the user control from the first state to the second state removes air from a chamber within the user control, wherein the restitution limiter permits air intake into the chamber at a rate such that the user control returns to the first state at the end of the time period.

16. The breathing system of claim 15, wherein the control means includes an expansile member into which gas is located by operation of the user control, thereby to expand the expansile member, wherein the expansile member acts on the substance to drive the substance at the flow restrictor.

17. The breathing system of claim 14, wherein the user control is biased to the first state.

18. The breathing system of claim 1, wherein the contained gas is linked to the substance in the reservoir via an intermediary member that the contained gas causes to push against the substance, thereby to drive the substance to the vaporiser.

19. The breathing system of claim 1, further comprising:
a patient airway interface mountable on the patient so that inhaling and exhaling occur through the patient airway interface repetitively and consecutively.

20. The breathing system of claim 19, wherein the patient airway interface is mountable on the patient to be retained in place indefinitely without use of hands of the patient; and/or
wherein, in use of the breathing system, the substance adder is fixedly mounted on the patient airway interface; and/or
wherein the control apparatus, other than the user control, in use of the breathing system, is fixedly mounted on the patient airway interface.

21-23. (canceled)

24. The breathing system of claim 1, wherein the substance is an analgesic, anaesthetic and/or sedative substance.

25. The breathing system of claim 1, wherein the pressure reduction valve is configured to enable loss of the pressurised gas at a rate that is less than a rate at which the control apparatus can pressurise the gas contemporaneously with pressurising of the gas by the control apparatus.

26. A breathing system arranged for a patient to breathe through continuously and to inhale a substance from during breathing, comprising:
a substance adder configured to add for adding the substance to gas to be inhaled, comprising:
a reservoir for containing the substance in a liquid form;
a vaporiser for vaporising the substance;
a control apparatus configured to pressurise contained gas, wherein the contained gas is linked to the substance in the reservoir to drive the substance to the vaporiser, the control apparatus comprising a pressure reduction means for enabling loss of the pressurised gas at a rate that is less than a rate at which the control apparatus can pressurise the gas contemporaneously with pressurising of the gas by the control apparatus.
